(19) United States
(12) Patent Application Publication
(10) Pub. No.: US 2011/0046522 A1
(43) Pub. Date: Feb. 24, 2011

(54) Ultrasound Energy Delivery Assembly

(75) Inventors: Huey Chan, San Jose, CA (US); Sean McFerran, Newark, CA (US); Stephen Porter, Oakland, CA (US); Del Kjos, Pleasanton, CA (US); Steve Forcucci, Winchester, MA (US); David Constantine, Andover, MA (US); Jeffrey J. Vaitekunas, Fairview, PA (US); Katie Kane, Salem, MA (US); Mark Hamm, Lynnfield, MA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED INC., Maple Grove, MN (US)

(21) Appl. No.: 12/853,706

(22) Filed: Aug. 10, 2010

Related U.S. Application Data
(60) Provisional application No. 61/235,984, filed on Aug. 21, 2009.

(51) Int. Cl. A61N 7/00

(52) U.S. Cl. 601/2

(57) ABSTRACT

In a first embodiment, an ultrasound energy delivery assembly includes a waveguide and a catheter having a capture member. The capture member extends radially inward from an interior surface of the catheter into a lumen of the catheter and is configured to retain the enlarged distal tip so that the enlarged distal tip is temporarily prevented from moving proximally. In a second embodiment, an ultrasound energy delivery assembly includes a waveguide and a sheath covering at least a portion of the waveguide. In a third embodiment, an ultrasound energy delivery assembly includes a waveguide and a dual lumen catheter having a capture member. In a fourth embodiment, an ultrasound energy delivery assembly includes a waveguide and a catheter having a proximal waveguide lumen and a proximal guide wire lumen that merges with the proximal waveguide lumen at their respective distal ends to form a distal lumen. In a fifth embodiment, an ultrasound energy delivery assembly includes a waveguide and a catheter having a helical spring tip disposed over a distal section of the waveguide. In a sixth embodiment, an ultrasound energy delivery assembly includes a waveguide and a catheter extruded from a material such as expanded polytetrafluoroethylene. The catheter includes a braided layer having a multi-wire braided section and a single wire braided section formed from a braid wire from the multi-wire braided section.

### RELATED APPLICATION DATA

The present application claims the benefit under 35 U.S.C. §119 to U.S. provisional patent application Ser. No. 61/235,984, filed Aug. 21, 2009. The foregoing application is hereby incorporated by reference into the present application in its entirety.

### FIELD OF THE INVENTION

The disclosed inventions generally relate to assemblies for delivering ultrasound energy to tissue. More particularly, the disclosed inventions relate to assemblies for delivering ultrasound energy to tissue in the neurological vasculature.

### BACKGROUND

A stroke is a disease state where there is a disruption to the blood vessels supplying the neurological vasculature. This lack of blood flow (Ischemia) may be due to rapid onset (acute) of thrombosis (formed blood clot), embolism (transient blood clot), and/or hemorrhagic event (bleeding). According to the ASA (American Stroke Association), there were around 780,000 stroke cases in 2008. Of these stroke cases, around 180,000 cases were recurring and around 600,000 were new. Of these stroke cases, 13% were Hemorrhagic and 87% were Ischemic.

Ischemia is a disease state where there is decreased arterial blood flow and oxygenation to the brain due to an obstruction or narrowing of the supplying artery. This results in an infarction (cell death) of the tissue in the area. In order to treat this disease state the objective of an intervention therapy is to reestablish blood flow to the infarct area. Currently, there are limited interventional devices to treat this disease state in the neurological vasculature. One approach is to use ultrasound technology to induce cavitations to break-up the fiber structure within a clot in the peripheral vasculature, thereby ablating the clot. This ultrasound treatment is delivered through a wire, which can be called a “waveguide,” and oscillates at a frequency that does not harm adjacent healthy tissue.

Most ultrasound delivery assemblies also include single lumen configuration micro-access catheters that track directly over a guide wire and are extremely flexible and low profile to facilitate access to the extremely tortuous neurological vasculature. Current procedures for treating the neurological vasculature require access across the lesion or site of interest by keeping the guide wire or micro-access catheter positioned across the lesion.

Perceived problems with current catheter design include the need for an extremely flexible material that maintains desirable properties even in dual-lumen micro-access catheter. These desirable properties include, but are not limited to, high tensile strength, low friction coefficient, and extrudability in the small sizes. It would also be desirable to eliminate or minimize shifting of the waveguide within the micro-access catheter, and to improve the capability to create a passage across a lesion.

### SUMMARY

In one embodiment, an ultrasound energy delivery assembly includes a waveguide and a catheter. The waveguide includes an active zone configured to deliver ultrasound energy in a radial direction, and an enlarged distal tip. The catheter includes an inner lubricious layer, a middle reinforcement layer disposed outside of the inner lubricious layer, an outer polymer layer disposed outside of the middle reinforcement layer, an active zone window configured to allow passage of ultrasound energy, and a capture member. The capture member extends radially inward from an interior surface of the catheter into a lumen of the catheter and is configured to retain the enlarged distal tip so that the enlarged distal tip is prevented from moving proximally. The catheter also includes a closed or significantly reduced inner-diameter distal end, such that the waveguide is prevented from moving distally beyond the closed or significantly reduced inner-diameter distal end and the waveguide is temporarily secured in the catheter when the waveguide is disposed in the catheter with the enlarged distal tip distal of the capture member. Also, the active zone is positioned in the active zone window when the waveguide is disposed in the catheter with the enlarged distal tip distal of the capture member. The middle reinforcement layer includes a first wire coil having variable pitch and at least one additional wire coil disposed radially outside of the first wire coil. The catheter also includes an axial wire disposed in the outer polymer layer and configured to strengthen the catheter. In alternative embodiments, the catheter also includes at least one additional active zone window. In other embodiments, the catheter also includes a guide wire tip disposed on a distal end of the catheter and configured to create an opening for the catheter through a tissue. The catheter also includes a plurality of marker bands. The assembly can also include a vacuum device configured to collect and remove clot fragments.

In another embodiment, an ultrasound energy delivery assembly includes a waveguide and a sheath covering at least a portion of the waveguide. The waveguide has a waveguide proximal section and a waveguide distal section, which includes an active zone configured to deliver ultrasound energy in a radial direction. The sheath includes a sheath proximal section secured to the waveguide proximal section, a sheath distal section configured to cover the waveguide distal section and the active zone, and an active zone window configured to allow passage of ultrasound energy. The sheath proximal section is a high density polyethylene tube, the sheath distal section is a low density polyethylene tube, and the high density polyethylene tube and the low density polyethylene tube are joined end to end. In alternative embodiments, the catheter also includes at least one additional active zone window. The assembly can also include a vacuum device configured to collect and remove clot fragments.

In yet another embodiment, an ultrasound energy delivery assembly includes a waveguide and a catheter. The waveguide includes an active zone configured to deliver ultrasound energy in a radial direction and an enlarged distal tip. The catheter includes a waveguide lumen configured to contain the waveguide and a guide wire lumen configured to contain a guide wire. The waveguide lumen includes a capture member extending radially inward from an interior surface of the catheter into a lumen of the catheter. The waveguide lumen also includes an active zone window configured to allow passage of ultrasound energy. The capture member is configured to retain the enlarged distal tip so that the enlarged distal tip is prevented from moving proximally. The catheter also includes a plurality of marker bands. The assembly can also include a vacuum device configured to collect and remove clot fragments.

In still another embodiment, an ultrasound energy delivery assembly includes a waveguide and a catheter. The waveguide includes an active zone configured to deliver ultrasound energy in a radial direction and an enlarged distal tip. The catheter includes a proximal waveguide lumen and a proximal guide wire lumen that merges with the proximal waveguide lumen at their respective distal ends to form a distal lumen. The distal lumen includes an active zone window configured to allow passage of ultrasound energy. The catheter also includes an axial wire disposed approximately opposite the active zone window and configured to strengthen the catheter. The assembly can also include a vacuum device configured to collect and remove clot fragments.

In another embodiment, an ultrasound energy delivery assembly includes a waveguide and a catheter. The waveguide includes a proximal section, a distal section, and an enlarged distal tip. The catheter includes an inner lubricious layer, a middle reinforcement layer disposed over the proximal section and outside of the inner lubricious layer, a helical spring tip disposed over the distal section and outside of the inner lubricious layer, and an outer polymer layer disposed outside of the inner lubricious layer. The middle reinforcement layer includes a first wire coil having variable pitch and at least one additional wire coil disposed radially outside of the first wire coil. The catheter also includes a proximal marker band and a distal marker band. A distal end of the helical spring tip is secured to the enlarged distal tip of the waveguide by the distal marker band. The assembly can also include a vacuum device configured to collect and remove clot fragments.

In yet another embodiment, an ultrasound energy delivery assembly includes a waveguide and a catheter. The waveguide includes an active zone configured to deliver ultrasound energy in a radial direction. The catheter is extruded from expanded polytetrafluoroethylene and includes a waveguide lumen configured to contain the waveguide and a guide wire lumen configured to contain a guide wire. The waveguide lumen includes an active zone window configured to allow the active zone of the waveguide to exit and re-enter the catheter. The catheter includes a braided layer having a multi-wire braided section covering a first portion of the catheter, and a single wire braided section covering a second portion of the catheter. One braid wire from the multi-wire braided section extends to and forms the single wire braided section. The catheter also includes a polymer outer layer covering the braided layer. The catheter also includes an axial wire configured to strengthen the catheter. In alternative embodiments, the catheter also includes at least a second active zone window. The assembly can also include a vacuum device configured to collect and remove clot fragments.

### BRIEF DESCRIPTION OF THE DRAWINGS

Referring now to the drawings in which like reference numbers represent corresponding parts throughout, and in which:

FIG. 1 is a side perspective view of an ultrasound energy delivery assembly in accordance with one embodiment of the disclosed inventions.

FIG. 2 is a midline longitudinal cross sectional view of the ultrasound energy delivery assembly in FIG. 1.

FIGS. 3 and 4 are detailed cross sectional views through the lines 3-3 and 4-4 in FIG. 2, respectively.

FIG. 5 is a midline longitudinal cross sectional view of an ultrasound energy delivery assembly in accordance with another embodiment of the disclosed inventions.

FIG. 6 is a side view of an ultrasound energy delivery assembly in accordance with another embodiment of the disclosed inventions.

FIGS. 7 and 8 are midline longitudinal cross sectional views of the ultrasound energy delivery assembly in FIG. 6.

FIG. 9 is a detailed cross sectional view through the line 9-9 in FIG. 8.

FIG. 10 is a side view of an ultrasound energy delivery assembly in accordance with another embodiment of the disclosed inventions.

FIG. 11 is a midline longitudinal cross sectional view of the ultrasound energy delivery assembly in FIG. 10.

FIG. 12 is a detailed cross sectional view through the line 12-12 in FIG. 11.

FIG. 13 is a side view of an ultrasound energy delivery assembly in accordance with another embodiment of the disclosed inventions.

FIG. 14 is a midline longitudinal cross sectional view of the ultrasound energy delivery assembly in FIG. 13.

FIG. 15 is a side view of an ultrasound energy delivery assembly in accordance with another embodiment of the disclosed inventions.

FIG. 16 is a midline longitudinal cross sectional view of the ultrasound energy delivery assembly in FIG. 15.

FIG. 17 is a side view of an ultrasound energy delivery assembly in accordance with another embodiment of the disclosed inventions.

FIG. 18 is a midline longitudinal cross sectional view of the ultrasound energy delivery assembly in FIG. 17.

FIG. 19 is a side view of an ultrasound energy delivery assembly in accordance with another embodiment of the disclosed inventions.

FIG. 20 is a midline longitudinal cross sectional view of the ultrasound energy delivery assembly in FIG. 19.

FIG. 21 is a midline longitudinal cross sectional view of an ultrasound energy delivery assembly in accordance with another embodiment of the disclosed inventions.

FIG. 22 is a side perspective view of the ultrasound energy delivery assembly in FIG. 1.

FIG. 23 is a midline longitudinal cross sectional view of the ultrasound energy delivery assembly in FIG. 22.

FIGS. 24 and 25 are detailed cross sectional views taken at lines 24-24 and 25-25, respectively, in FIG. 23.

### DETAILED DESCRIPTION OF THE ILLUSTRATED EMBODIMENTS

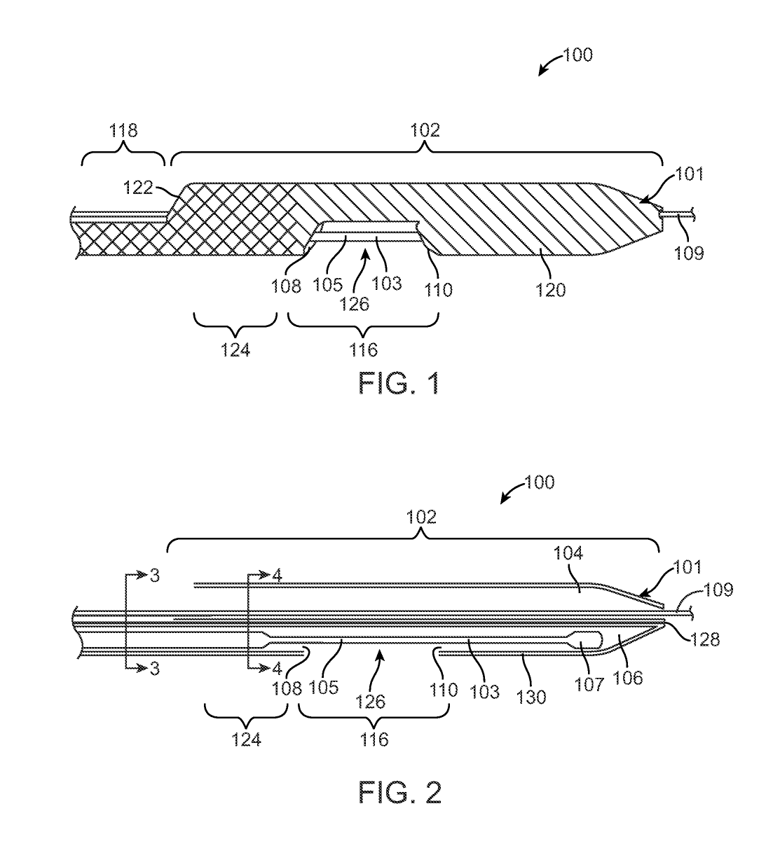

FIG. 1 shows an ultrasound energy delivery assembly 100 for neurological/stroke thrombectomy, including a monorail rapid exchange micro-access catheter 101. The micro-access catheter 101 can be used for any application with extreme tortuosity that requires a highly flexible, dual-lumen catheter segment. The assembly 100 also includes a waveguide 103, which has an active zone 105 and an enlarged distal tip 107. The active zone 105 is configured to deliver ultrasound energy in a radial direction. The assembly 100 is mounted on a standard guide wire 109 when in use.

The micro-access catheter 101 has two lumens that are formed in a single extrusion, a guide wire lumen 104 and a waveguide lumen 106. The guide wire lumen 104 is configured to contain a standard guide wire 109. The waveguide lumen 106 is skived away or closed off using heat and heat shrinkable tubing that is subsequently trimmed, with skives 108, 110 at the proximal and distal ends of the active zone window 116 to allow the active zone 105 of waveguide 103 to allow ultrasound treatment energy to access the lesion site.

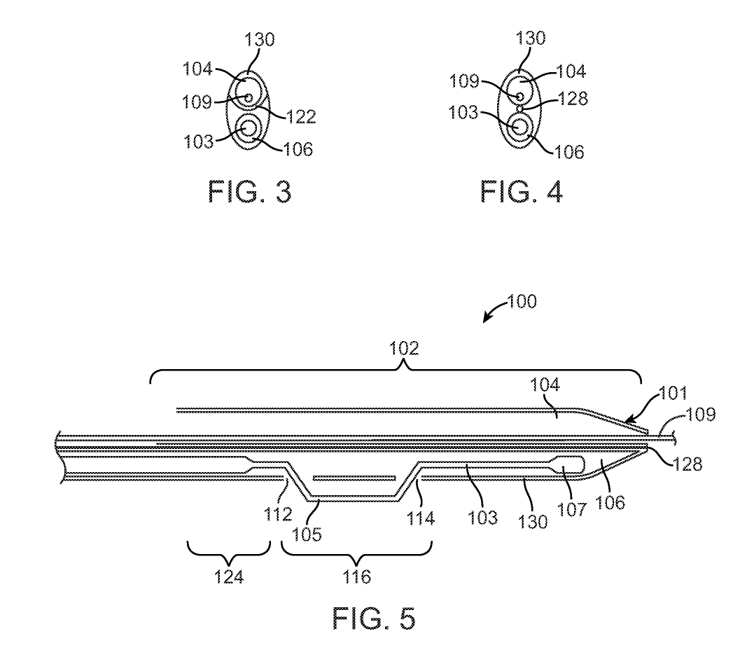

As shown in FIGS. 2, 3, and 4, the distal dual lumen or “monorail” segment 102 of the micro-access catheter 101 incorporates a single extrusion with dual lumens 104, 106. This dual lumen configuration allows the micro-access catheter 101 to be threaded onto a guide wire 109 and delivered to a target site, leaving the waveguide lumen 106 available to carry a waveguide 103.

Another embodiment of the active zone, shown in FIG. 5, includes two skived or cored holes 112, 114 with the waveguide lumen 106 between them left open for simplicity, and to retain column strength in the catheter active zone window 116 to support the thin waveguide active zone 105. The active zone 105 exits and re-enters the catheter 101 through the holes 112, 114.

A preferred embodiment utilizes a specialized expanded PTFE (ePTFE) Teflon material that retains the desirable properties of PTFE Teflon such as (but not limited to) low friction coefficient, high tensile strength, and biocompatibility. ePTFE differs from regular PTFE Teflon in that it is expanded during the extrusion process, which renders the material extremely flexible while retaining its desirable properties.

As shown in FIG. 1, a braided, PTFE lined proximal shaft 118 provides adequate column strength and efficiently transfers torque to the distal monorail segment 102, with braid extending onto the monorail segment 102, and braid wires 120 from one winding direction (clock-wise or counter-clock-wise) extend to near the distal tip to transmit torque and kink resistance to the distal tip. A thin-walled tube of Pebax or PTFE Teflon encapsulates the braid and inner extrusions(s), and serves to create a more gradual transition between proximal shaft and monorail segments. An angled lap joint 122 between proximal shaft and distal monorail segments 118, 102 also helps gradually transition stiffness between the two segments. Extending the braid wires over the joint 122 and onto the dual-lumen exchange section 124 (proximal end of monorail segment 102) is another method that gradually transitions stiffness. The active zone window is reinforced with an axial wire 128.

This micro-access catheter configuration allows a clinician to use currently accepted catheterization procedures, keeping access across the lesion with a standard 0.014″ diameter guide wire 109 and exchanging the catheter used to access the lesion for the micro-access catheter 101, or alternatively, using the micro-access catheter 101 to access the lesion.

The polymer outer layer 130 that encapsulates the braid wires extending over the distal end of the served wires is made from one of the soft grades of Pebax, such as (but not limited to) 3533 or 4033, used with a coating, such as a hydrophilic coating, to reduce frictional drag, or PTFE or ePTFE to eliminate the hydrophilic coating.

In one embodiment, approximate dimensional ranges for the ePTFE monorail segment are as follows:

| Monorail length | 10 cm-40 cm |
|---|---|
| Monorail OD | 0.033″-0.040″ |
| Waveguide lumen ID | 0.004″-0.011″ |
| Guide wire lumen ID | 0.016″-0.018″ |
| Outer Jacket wall thickness | 0.0005″-0.002″ |

Alternative embodiments can incorporate a vacuum device attached to the delivery catheter. The vacuum device allows the collection and removal of clot fragments. This design feature would also allow the capture of clot through aspiration.

Alternate materials that could suffice for the distal, dual-lumen segment of the neuron/stroke catheter include but are not limited to; PEBAX 3533; EVA or any highly flexible biocompatible polymer that is extrudable in small cross-sections. Further, two separate thin-walled extrusions made from a highly flexible polymer could replace the dual-lumen ePTFE extrusion if they were loosely tied together to maintain flexibility.

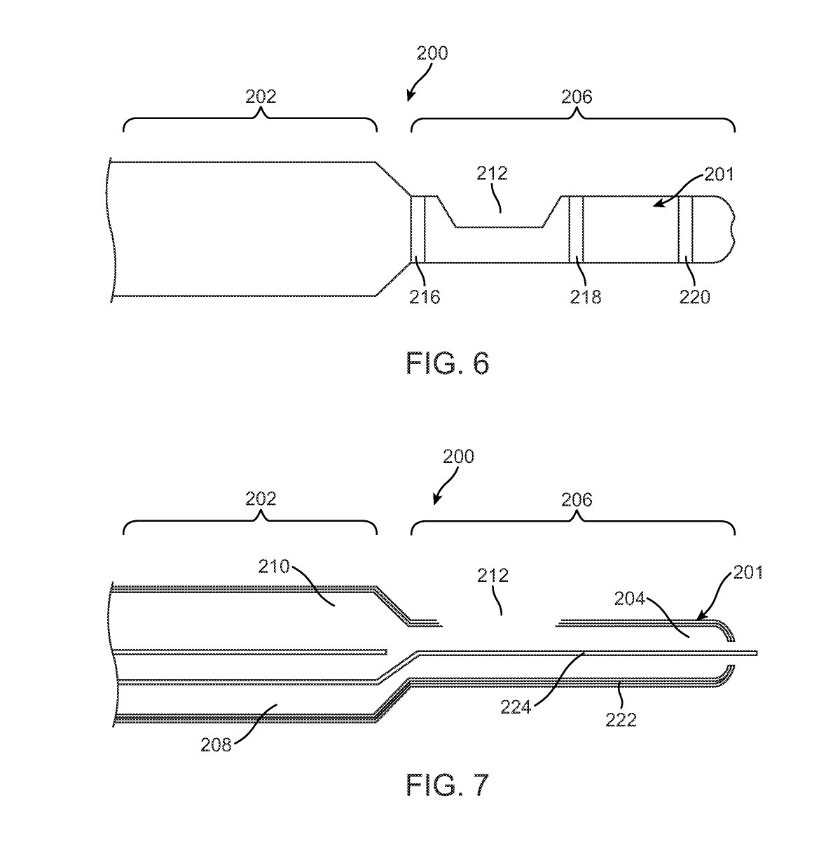

In another embodiment, shown in FIGS. 6-9, an ultrasound energy delivery assembly 200, includes a micro-access catheter 201, a waveguide 214, which has an active zone 205 and an enlarged distal tip 207. The active zone 205 is configured to deliver ultrasound energy in a radial direction. During certain phases of its use, the assembly 200 is mounted on a standard guide wire 224 as described below.

The micro-access catheter 201 has a dual lumen proximal shaft 202 that tapers into a single distal lumen 204 at the distal shaft 206. This creates a low distal profile for distal access. One of the two proximal lumens is a guide wire lumen 208, and the other proximal lumen is a waveguide lumen 210. This configuration allows for rapid exchange of the guide wire 224 and the waveguide 214 during use with only minimal movement of the catheter 201.

The active zone window 212 at the distal tip is created by removing distal shaft material to expose the active zone of the waveguide 214. Proximal, middle, and distal markers bands 216, 218, 220 are embedded inside the distal shaft 206, as shown in FIG. 6. The active zone window 212 is reinforced with a material, such as (by way of non-limiting example) a Nitinol wire 222 for kink resistance, as shown in FIG. 9. An atraumatic tip is created by fusing the polymer tubing at the distal end of the distal shaft 206. The proximal shaft 202 can be reinforced for kink resistance and to provide proximal push. The distal shaft 206 can be selectively reinforced with variable pitch for flexibility.

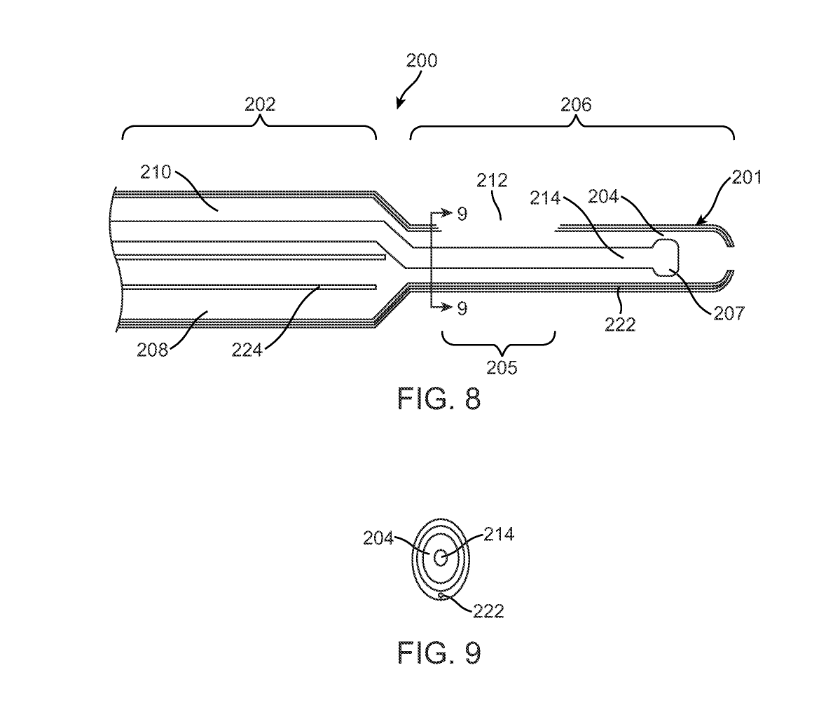

To access to the destination site, the guide wire 224 is advanced out of the distal tip through the single distal lumen 204, as shown in FIG. 7. After the micro-access catheter 201 has arrived at its destination, the guide wire 224 is retracted inside the distal end of the guide wire lumen 208, as shown in FIG. 8. The waveguide 214 is advanced inside the single distal lumen 204 until it reaches the proximal end of the distal marker band 220. This positions the active zone 205 of the waveguide 214 adjacent the active zone window 212 for treatment.

It should be appreciated that although FIGS. 5, 7, and 8 depict the waveguide employing sharp bends, the illustrations are for purposes of clarity and not limitation. It is contemplated within the scope of the disclosed inventions that the waveguide bends would typically be gradual so that the ultrasonic energy is not disrupted by a tight bend-radius. In particular, the embodiments depicted in FIGS. 5, 7 and 8, among others, are compressed in length scale for clarity, and actual the actual waveguide bends typically are more gradual.

Alternative embodiments incorporate a vacuum device attached to the delivery catheter to allow collection and removal of clot fragments. This design feature may also possibly allow the capture of clot through aspiration.

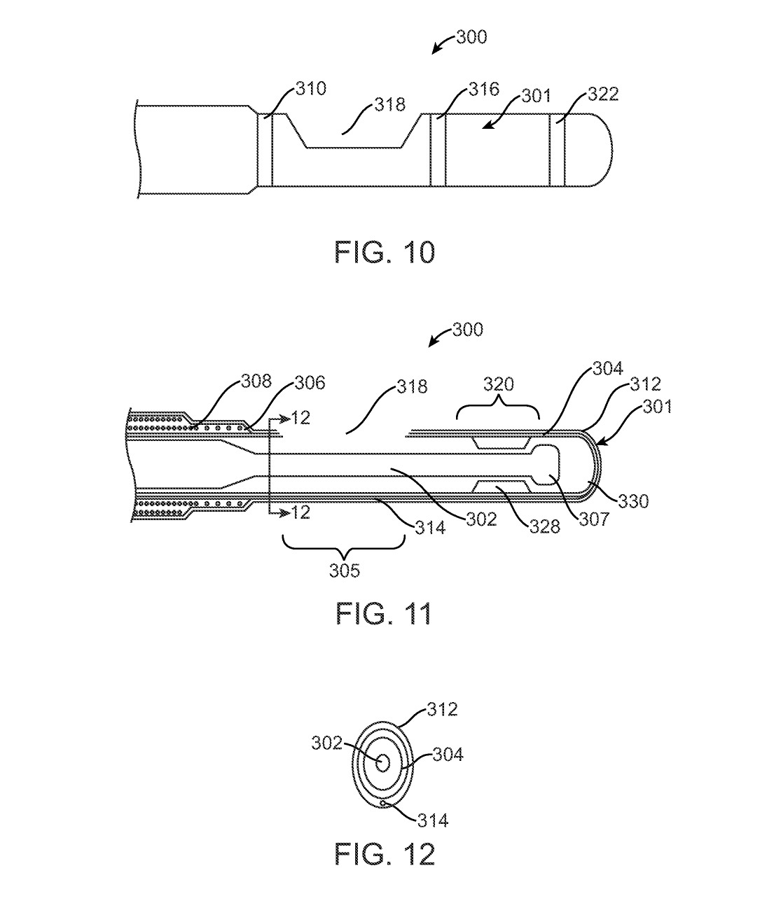

In another embodiment shown in FIGS. 10-12, an ultrasound energy delivery assembly 300 includes a single lumen reinforced catheter 301 that encapsulates a waveguide 302 which has an active zone 305 and an enlarged distal tip 307. The active zone 305 is configured to deliver ultrasound energy in a radial direction.

The inner layer 304 of the catheter 301 can be constructed with a lubricious polymer such as PTFE. The lubricious inner layer 304 provides for low coefficient of friction as the waveguide 302 moves longitudinally at the proximal section. A (preferably) metal reinforcement layer 306 is wound over the lubricious inner layer 304 using a material such as (but not limited to) stainless steel or Nitinol wire 308. The reinforcement layer 306 has variable pitch for flexibility at the distal tip and provides maximum column support at the proximal shaft. The proximal reinforcement layer 306 could be composed of multiple layers of coils to provide maximum stiffness. The reinforcement layer 306 terminates distal of the proximal marker 310. The outer layer 312 is laminated over the reinforcement layer 306. The polymer outer layer 312 can be composed of material of different stiffness to create flexibility at the distal tip and stiffness at the proximal shaft.

The distal tip is reinforced with a wire 314 such as Nitinol, as shown in FIG. 12. The wire is encapsulating between the inner lubricious and outer polymer layer 304, 312 and is secured in place with the proximal 310 and mid marker band 316. Opposite to the wire, the active zone window 318 is created by removing the distal shaft material.

A capture tip section 320 is formed as a part of the catheter 301 using a polymer to form a capture member 328. The capture member 328 extends radially inward from an interior surface of the catheter 301. The capture member 328 allows the passage of the enlarged waveguide distal tip 307 distally under pressure, but prevents it from backing out under normal pressures applied during treatment. The distal end 330 of the catheter 301 prevents the waveguide 302 from moving distally. Therefore, once the enlarged waveguide distal tip 307 is moved distal of the capture member 328, the capture member 328 and the distal end 330 of the catheter 301 cooperate to temporarily secure the waveguide 302 in place. In this position, the active zone 305 of the waveguide 302 is positioned adjacent the active zone window 318 of the catheter 301.

The distal section of the catheter may be sized anywhere from 0-50 cm, and in one embodiment is around 0-10 cm and can be tapered. Just distal to the mid marker band 316 in the distal section is a distal marker band 322 that aids in the visualization of the distal section during treatment. An atraumatic tip can be formed by fusing the polymer tubing at the distal end of the shaft.

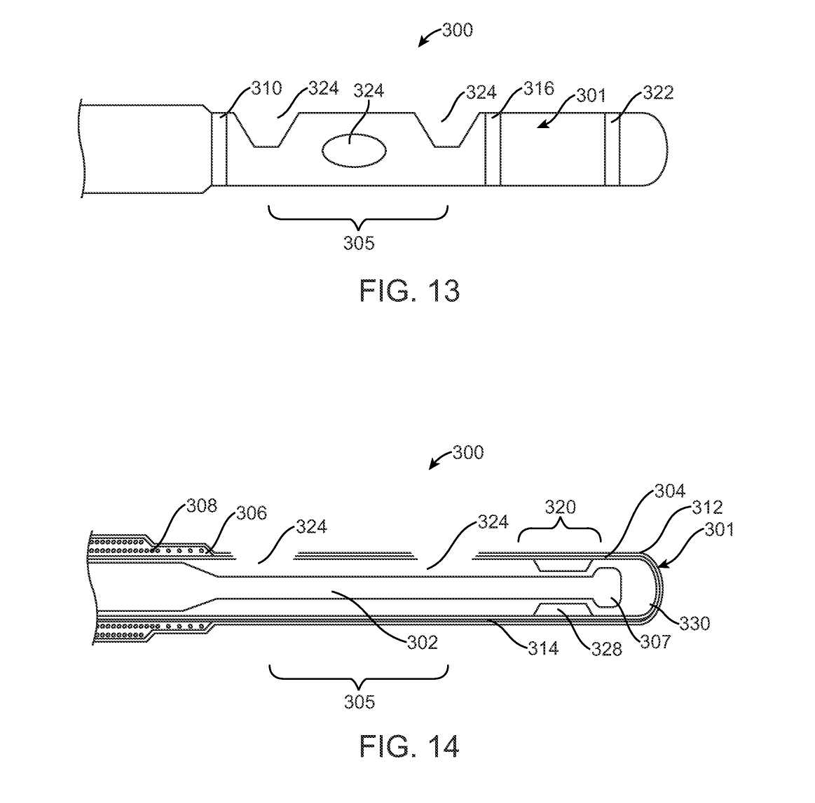

In a similar embodiment, shown in FIGS. 13 and 14, the active zone window 316 is replaced by a plurality of smaller active zone windows 324 to strengthen the active zone of the catheter 301.

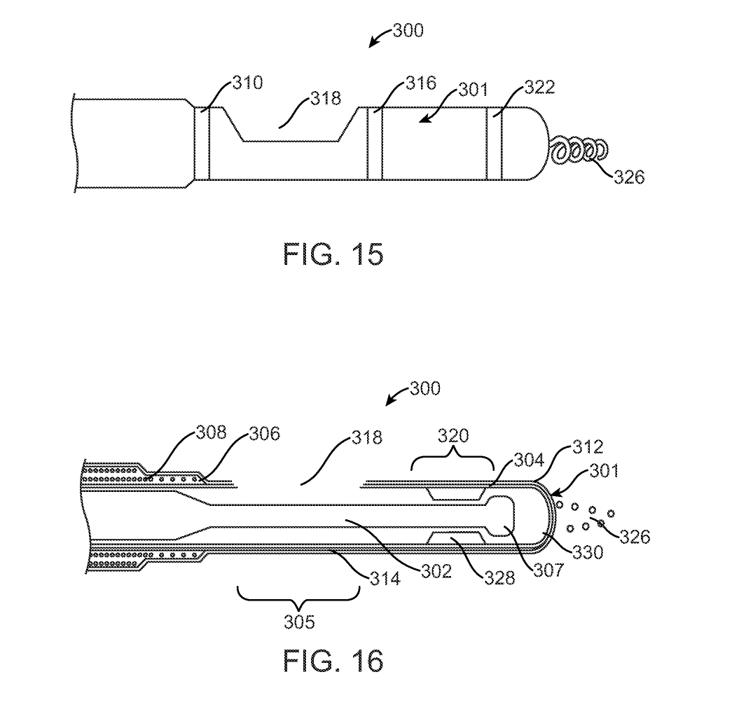

In a similar embodiment, shown in FIGS. 15 and 16, a guide wire tip 326 is attached to the distal end of the micro-access catheter 301. The guide wire tip 326 forms a corkscrew shape to create a passage through a lesion (blood clot).

Alternative embodiments incorporate a vacuum device attached to the delivery catheter to allow collection and removal of clot fragments, and also to possibly allow the capture of clot through aspiration.

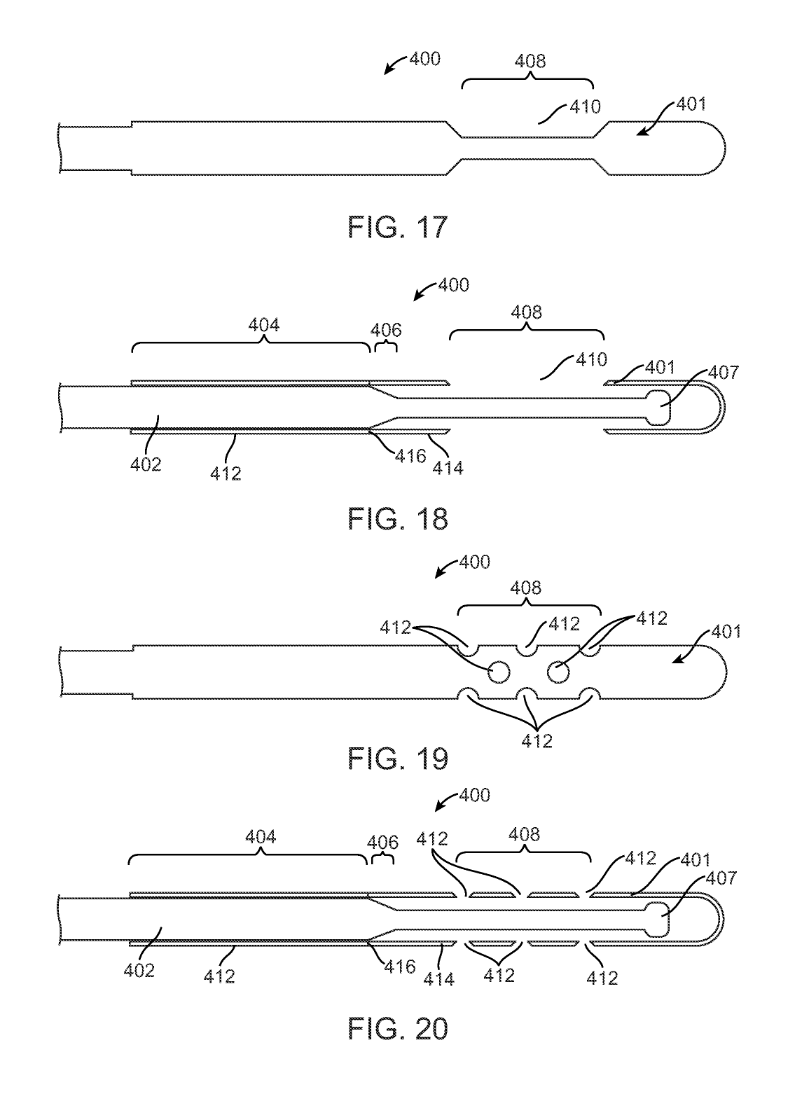

In another embodiment, shown in FIGS. 17 and 18, an ultrasound energy delivery assembly 400 includes a waveguide 402 encapsulated by a sheath 401. The waveguide 402 has an active zone 408 and an enlarged distal tip 407. The active zone 408 is configured to deliver ultrasound energy in a radial direction.

A proximal section 412 of the sheath 401 is directly mounted over a proximal straight barrel section 404 of the waveguide 402. This can be accomplished by heat shrinking polymer tubing, such as (but not limited to) high density polyethylene (HDPE) tubing, over the proximal section 404 of the waveguide 402. The tapered section 406 and active zone 408 of the waveguide 402 is protected by a distal section 414 of the sheath 401. The distal section 414 can be formed with polymer tubing such as (but not limited to) low density polyethylene (LDPE) tubing. The proximal section 412 and the distal section 414 of the sheath 401 are joined end to end at a joint 416. The active zone window 410 is created by removing material to expose the active zone 408 of the waveguide 402.

In a similar embodiment, shown in FIGS. 19 and 20, the active zone window 410 is replaced by a plurality of smaller active zone windows 412 to strengthen the sheath 401 near the active zone 408.

Alternative embodiments incorporate a vacuum device attached to the delivery catheter. The vacuum device can be included to allow collection and removal of clot fragments, and also to possibly allow for the capture of clot through aspiration.

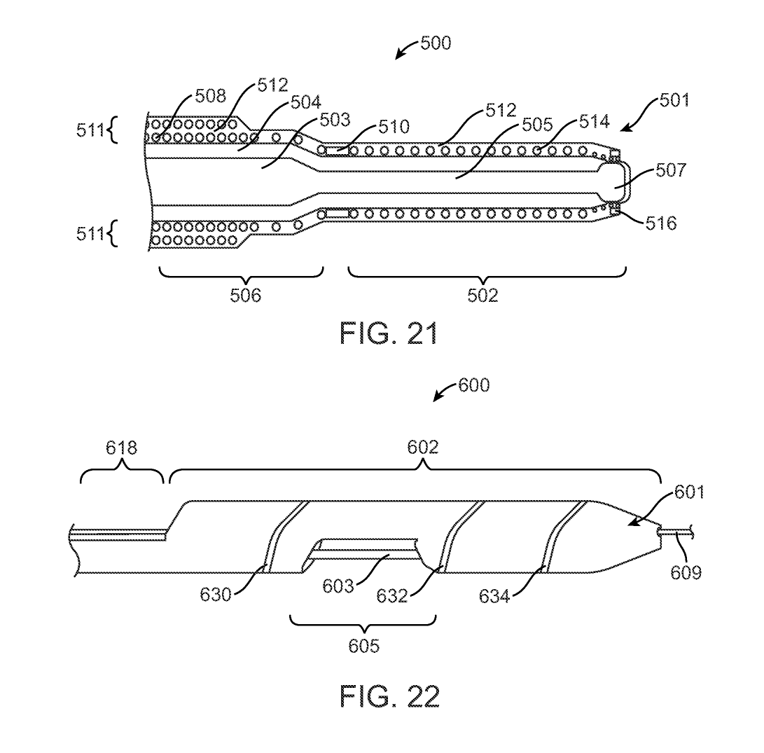

In an embodiment shown in FIG. 21, FIG. 1 an ultrasound energy delivery assembly 500 includes a single lumen reinforced catheter 501 and a waveguide 503, which has an active zone 505 and an enlarged distal tip 507.

The catheter 501 has a distal section 502 and a proximal section 506. The inner layer 504 of the catheter 501 is made of a lubricious polymer such as (but not limited to) polytetrafluoroethylene (PTFE). The lubricious inside layer 504 provides for low coefficient of friction as the waveguide moves longitudinally. A metal reinforcement layer 511 is wound over the lubricious layer 504 using a material such as stainless steel or Nitinol wire 508. The wound reinforcement layer 511 has variable pitch for maximum column support at the proximal section 506. The proximal end of the reinforcement layer 511 is composed of multiple layers of coils to provide maximum stiffness. The reinforcement layer 511 is terminated at the proximal marker 510. The outer layer 512 is laminated over the reinforcement layer 511. The polymer outer layer 512 can be composed of different durometers of polymer such as (but not limited to) Pebax to create flexibility at the distal section 502 and stiffness at the proximal section 506.

A helical spring 514 is mounted over the distal section 502 of the catheter 501 and outside of the inner layer 504. The helical spring 514 can be secured by one or more of the marker bands 510, 516. The polymer outer layer 512 is laminated over the helical spring 514 and the marker bands 510, 516. PTFE is loosely placed over the distal section 502 of the assembly 500. The distal end of the helical spring 514 is secured over the PTFE of the polymer outer layer 512 and the tip of the waveguide 503 by crimping the distal marker band 516 over the tip of the waveguide 503. The polymer tip is melted over the distal marker band 516 to form an atraumatic tip.

Alternative embodiments may incorporate a vacuum device attached to the delivery catheter to allow for collection and removal of clot fragments, as well as to possibly allow for the capture of clot through aspiration.

In an embodiment shown in FIGS. 22-25, an ultrasound energy delivery assembly 600 includes a monorail rapid exchange micro-access catheter 601 and a waveguide 603, which has an active zone 605 and an enlarged distal tip 607. The active zone 605 is configured to deliver ultrasound energy in a radial direction. The assembly 600 is mounted on a guide wire 609 when in use.

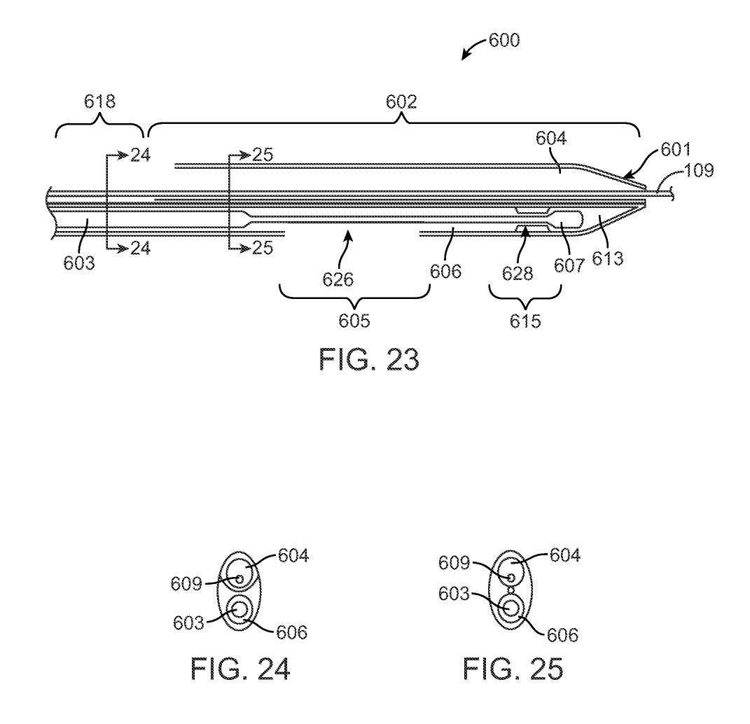

The catheter 601 includes a single lumen proximal shaft 618 and a dual lumen distal shaft 602. In use, the waveguide lumen 606 contains the waveguide 603 which continues to the distal tip 613 of the micro-access catheter 601. As shown in FIGS. 22 and 23, the proximal end of the guide wire lumen 604 ends 20-100 cm from the distal tip 613 of the micro-access catheter 601 and accepts guide wires 609 such as (but not limited to) a 0.014″ guide wire. The active zone 605 of the waveguide 603 is exposed through the active zone window 626. The enlarged distal tip 607 of the waveguide 603 is captured at the distal tip 613 of the micro-access catheter 601 by a capture tip 615.

A capture tip 615 is formed as a part of the catheter 601 using a polymer to form a capture member 628. The capture member 628 extends radially inward from an interior surface of the catheter 601. The capture member 628 allows the passage of the enlarged waveguide distal tip 607 distally under pressure, but prevents it from backing out under normal pressures applied during treatment. The distal tip 613 of the catheter 601 prevents the waveguide 603 from moving distally. Therefore, once the enlarged waveguide distal tip 607 is moved distal of the capture member 628, the capture member 628 and the distal tip 613 of the catheter 601 cooperate to temporarily secure the waveguide 603 in place. In this position, the active zone 605 of the waveguide 603 is positioned adjacent the active zone window 626 of the catheter 601.

As shown in FIG. 22, markers bands 630, 632, 634 are embedded inside the tubing. The proximal and middle marker bands 630, 632 on the distal shaft 602 are used to visualize of the active zone window 626. The distal marker band 634 is used to visualize the distal end of the micro-access catheter 601. An atraumatic tip is formed by fusing the polymer tubing at the distal end of the micro-access catheter 601. The guide wire lumen can be reinforced with variable pitch for flexibility.

Alternative embodiments can incorporate a vacuum device attached to the delivery catheter to allow for collection and removal of clot fragments. This design feature may also possibly allow for the capture of clot through aspiration.

While various embodiments of the disclosed inventions have been shown and described, they are presented for purposes of illustration, and not limitation. Various modifications may be made to the illustrated and described embodiments without departing from the scope of the disclosed inventions, which is to be limited and defined only by the following claims and their equivalents.

1. An ultrasound energy delivery assembly, comprising:
a waveguide, comprising:
an active zone configured to deliver ultrasound energy in a radial direction; and
an enlarged distal tip; and
a catheter, comprising:
an inner lubricious layer;
a middle reinforcement layer disposed outside of the inner lubricious layer;
an outer polymer layer disposed outside of the middle reinforcement layer;
an active zone window configured to allow passage of ultrasound energy; and
a capture member extending radially inward from an interior surface of the catheter into a lumen of the catheter, wherein the capture member is configured to retain the enlarged distal tip so that the enlarged distal tip is temporarily prevented from moving proximally.

2. The ultrasound energy delivery assembly of claim 1, the catheter further comprising a closed distal end, wherein the waveguide is prevented from moving distally beyond the closed distal end.

3. The ultrasound energy delivery assembly of claim 1, wherein the waveguide is temporarily secured in the catheter when the waveguide is disposed in the catheter with the enlarged distal tip distal of the capture member.

4. The ultrasound energy delivery assembly of claim 1, wherein the active zone is positioned in the active zone window when the waveguide is disposed in the catheter with the enlarged distal tip distal of the capture member.

5. The ultrasound energy delivery assembly of claim 1, the middle reinforcement layer comprising a first coil having variable pitch.

6. The ultrasound energy delivery assembly of claim 5, the middle reinforcement layer further comprising at least one additional coil disposed radially outside of the first coil.

7. The ultrasound energy delivery assembly of claim 1, the catheter further comprising an axial wire disposed in the outer polymer layer and configured to strengthen the catheter.

8. The ultrasound energy delivery assembly of claim 1, the catheter further comprising at least one additional active zone window.

9. The ultrasound energy delivery assembly of claim 1, the catheter further comprising a guide wire tip disposed on a distal end of the catheter and configured to create an opening for the catheter through a tissue.

10. The ultrasound energy delivery assembly of claim 1, the catheter further comprising a plurality of marker bands.

11. The ultrasound energy delivery assembly of claim 1, further comprising a vacuum device configured to collect and remove clot fragments.

12. An ultrasound energy delivery assembly, comprising:
a waveguide, comprising a waveguide proximal section and a waveguide distal section including an active zone configured to deliver ultrasound energy in a radial direction; and
a sheath covering at least a portion of the waveguide, comprising:
a sheath proximal section secured to the waveguide proximal section;
a sheath distal section configured to cover the waveguide distal section and the active zone; and
an active zone window configured to allow passage of ultrasound energy.

13. The ultrasound energy delivery assembly of claim 12, wherein the sheath proximal section comprises a high density material.

14. The ultrasound energy delivery assembly of claim 12, wherein the sheath distal section comprises a low density material.

15. The ultrasound energy delivery assembly of claim 12, wherein the sheath proximal section comprises a high density material, the sheath distal section comprises a low density material, wherein the respective high density and low density materials are joined end to end.

16. The ultrasound energy delivery assembly of claim 12, the catheter further comprising at least one additional active zone window.

17. The ultrasound energy delivery assembly of claim 12, further comprising a vacuum device configured to collect and remove clot fragments.

18. An ultrasound energy delivery assembly, comprising:
a waveguide, comprising:
an active zone configured to deliver ultrasound energy in a radial direction; and
an enlarged distal tip; and
a catheter, comprising:
a waveguide lumen configured to contain the waveguide, comprising:
a capture member extending radially inward from an interior surface of the catheter into a lumen of the catheter, wherein the capture member is configured to retain the enlarged distal tip so that the enlarged distal tip is temporarily prevented from moving proximally; and
an active zone window configured to allow passage of ultrasound energy; and
a guide wire lumen configured to contain a guide wire.

19. The ultrasound energy delivery assembly of claim 18, the catheter further comprising a plurality of marker bands.

20. The ultrasound energy delivery assembly of claim 18, further comprising a vacuum device configured to collect and remove clot fragments.

21-33. (canceled)
